(19) United States
(12) Patent Application Publication
(10) Pub. No.: US 2024/0342004 A1
(43) Pub. Date: Oct. 17, 2024

(54) Method for positioning an interpositional ophthalmological implant at a distance from the limbus

(71) Applicant: CILIATECH, Chavanod (FR)

(72) Inventors: Olivier BENOIT, EPAGNY METZ-TESSY (FR); Philippe SOURDILLE, GUERANDE (FR)

(21) Appl. No.: 18/610,836

(22) Filed: Mar. 20, 2024

(30) Foreign Application Priority Data
Mar. 23, 2023 (FR) 2302607

(51) Int. Cl. A61F 9/007; A61F 9/00

(52) U.S. Cl. CPC A61F 9/00781; A61F 9/0017

(57) ABSTRACT

A method is provided for inserting an ophthalmological implant between a sclera and a ciliary body in an eye of a patient. The method includes making at least one incision through the sclera in the eye of the patient forming at least one scleral edge and inserting the ophthalmological implant between the sclera and the ciliary body through the at least one incision. The method includes positioning the ophthalmological implant at a final position so that a distance between an anterior edge of the ophthalmological implant and a limbus of the eye of the patient is equal to or greater than 2 mm along an anteroposterior axis.

### RELATED APPLICATION

This application claims the benefit of French Patent Application 23 02607, filed on Mar. 21, 2023, the entirety of which is incorporated by reference.

### FIELD OF THE INVENTION

The invention relates to an interpositional ophthalmological implant intended to keep the sclera and the ciliary body permanently separated from one another in order to lower intraOcular pressure (IOP).

### DESCRIPTION OF THE RELATED ART

A human eye is usually split in two distinct anatomical volumes:
the anterior segment which contains the lens, the cornea, the ciliary body and the iris, and in between the corneal and the lens a space named “anterior chamber”,
the posterior segment with the vitreous and the retina.

The conjunctiva and the sclera (the white non-transparent part or the eye) covers mainly the posterior segment.

Glaucoma is a sight threatening disorder characterised by chronic IntraOcular Pressure (“IOP”) increase beyond the normal limits. The reason for this IOP increase lies—very schematically—in the difficulty for the aqueous humour to drain out of the anterior chamber.

Two ways of aqueous humour circulation are well known: the trabecular and the uveo-scleral outflows. Both of them-alone or in combination—can be the cause for the IOP increase, and both of them can be purpose for treating the disease.

Surgery is one way to get IOP under control. Several techniques exist, and they all consist into re-establishing a sustainable and controlled drainage of the aqueous humour from the AC to either the sub-conjunctival or the supraciliary/suprachoroidal spaces. This is obtained by incising tissues and creating artificial egresses, with two approaches:
ab-externo: the surgery is performed from the outside of the eye, the conjunctiva, sclera, corneal and other tissues may be incised or removed, or
ab-interno: the surgery is performed from the inside of the eye, specifically from the anterior chamber; an incision is performed in the cornea and the surgeon manipulates surgical tools from the inside of the anterior chamber.

Surgeries can be performed with or without implant. Implants are expected to prolong the IOP-lowering effect of the surgery by keeping openings and artificially created channels or aqueous egresses open and functional.

In the case of ab-externo approach, it exists one principal historical technique called “trabeculectomy”, which has been modified and improved in various sub-techniques such as “deep scleretomy” or “visco-canalostomy” for instance. All these techniques target the trabecular outflow. They require good manual skills and a long training before being mastered by the surgeons. And despite a long training and experience, such techniques still show many peri-operative risks and post-operative complications. This is why these techniques are regularly used by a limited number of surgeons, and dedicated to a certain category of patients.

Since a couple of years, some implants were invented in an attempt to simplify the surgical technique and make it available to all ophthalmologist surgeons. Experience has shown that if the surgical technique is simplified or the usual complications reduced, it usually comes with a limited IOP-lowering capacity or generates new long-term risks.

More recently another implant targeting the uveo-scleral outflow and using an ab-externo surgical approach has been introduced. In such case, the implant shall be positioned between the sclera and the ciliary body, in contact with the posterior side of the iris root.

If the relevant surgical technique is easier and safer than a regular trabeculectomy, the positioning of the device in contact with the posterior root of the iris remains a critical step and needs some trained hands. Moreover, the implant being positioned in a space where the surgeon has no visibility, the correct positioning of the device may not be always perfectly achieved.

### SUMMARY OF THE INVENTION

Given the foregoing, the present invention proposes a method for inserting an ophthalmological implant between a sclera and a uveal tissue in an eye of a patient, comprising:
providing an ophthalmological implant having an anterior edge intended to face the anterior chamber and an posterior edge opposite the anterior edge,
making at least one incision through the sclera in said eye of the patient forming at least one scleral edge,
lifting the at least one scleral edge;
inserting the ophthalmological implant between the sclera and the ciliary body through said at least one incision;
positioning the ophthalmological implant at a final position so that a distance between the anterior edge of the ophthalmological implant and a limbus of the eye of the patient is equal to or greater than 2 mm along an anteroposterior axis.

As previously mentioned, it was assumed that for a good IOP-lowering capability, the contact of the anterior edge of the implant with the posterior side of the iris root is necessary. In this regard, the prior art aims at positioning the ophthalmological implant as close as possible to the iris root.

A clinical study has shown that positioning an ophthalmological implant behind the iris root, particularly at a position where the anterior edge is at a distance equal or greater than 2 mm from the limbus, surprisingly allows to significantly reduce the IOP. The anterior edge of the ophthalmological implant is therefore not in contact with the iris root and not near the iris root (i.e. less than 1 or 2 mm from the limbus).

Furthermore, positioning the ophthalmological so that the anterior edge is at a distance equal or greater than 2 mm from the limbus reduces the complexity of the surgery by considering a visible anatomic reference (the limbus). Then, positioning the ophthalmological implant at a distance equal or greater than 2 mm reduces the uncertainty to have the implant contacting the iris root as in the prior art.

Hence, this specific position of the ophthalmological implant at a distance equal or greater than 2 mm allows to significantly reduce the IOP while reducing the complexity of the surgery.

According to an embodiment of said inserting method, said distance between the anterior edge of the ophthalmological implant and the limbus of the eye of the patient is equal to or greater than 2.5 mm along the anteroposterior axis.

According to an embodiment of said inserting method, said distance between the anterior edge of the ophthalmological implant and a limbus of the eye of the patient is equal to or lower than 5 mm along the anteroposterior axis.

According to an embodiment of said inserting method, said distance between the anterior edge of the ophthalmological implant and a limbus of the eye of the patient is equal to or lower than 4 mm along the anteroposterior axis.

According to an embodiment of said inserting method, said ophthalmological implant has an anteroposterior dimension between the anterior and the posterior edges equal to or greater than 500 μm.

According to an embodiment of said inserting method, the inserting step comprises the injection of a viscoelastic fluid between the sclera and the uveal tissues.

According to an embodiment of said inserting method, said at least one incision is radial along the anteroposterior axis.

According to an embodiment of said inserting method, said at least one incision comprises at least two incisions defining a first and a second incisions.

According to an embodiment of said inserting method, said first and second incisions are provided so that said ophthalmological implant is positioned between the first and second incisions when in the final position.

According to an embodiment of said inserting method, said first and second incisions are provided at an intermediate position of the ophthalmological implant so that said ophthalmological implant is inserted through said first or second incisions and then moved from said intermediate position wherein the ophthalmological implant is between the first and second incisions to said final position.

### BRIEF DESCRIPTION OF THE DRAWINGS

The invention is described in more detail below by way of the figures that show only one preferred embodiment of the invention.

FIG. 1 schematically shows a cross-section view of an eye of a patient wherein an ophthalmic implant is positioned between the sclera and the ciliary body at distance from the iris root;

FIG. 2 schematically shows a first embodiment of a method for positioning the ophthalmic implant through one incision into the sclera;

FIG. 3 schematically shows a second embodiment of said method for positioning the ophthalmic implant through two incisions into the sclera.

### DETAILED DESCRIPTION OF THE PREFERRED EMBODIMENTS

In the description which follows, the drawing figures are not necessarily to scale and certain features may be shown in generalized or schematic form in the interest of clarity and conciseness or for informational purposes. In addition, although making and using various embodiments are discussed in detail below, it should be appreciated that as described herein are provided many inventive concepts that may embodied in a wide variety of contexts. Embodiments discussed herein are merely representative and do not limit the scope of the invention. It will also be obvious to one skilled in the art that all the technical features that are defined relative to a process can be transposed, individually or in combination, to a device and conversely, all the technical features relative to a device can be transposed, individually or in combination, to a process.

The terms “comprise” (and any grammatical variation thereof, such as “comprises” and “comprising”), “have” (and any grammatical variation thereof, such as “has” and “having”), “contain” (and any grammatical variation thereof, such as “contains” and “containing”), and “include” (and any grammatical variation thereof such as “includes” and “including”) are open-ended linking verbs. They are used to specify the presence of stated features, integers, steps or components or groups thereof, but do not preclude the presence or addition of one or more other features, integers, steps or components or groups thereof. As a result, a method, or a step in a method, that “comprises”, “has”, “contains”, or “includes” one or more steps or elements possesses those one or more steps or elements but is not limited to possessing only those one or more steps or elements.

The present invention provides a method for inserting an ophthalmological implant between a sclera and a uveal tissue in an eye of a patient. The ophthalmologic implant may be a cyclodialysis implant or a permanent interpositional ophthalmologic implant.

For sake of clarity, the words “anterior edge” will be used further in the document to represent the proximal edge of an ophthalmologic implant, i.e. the part of the body of the implant the closest to the anterior chamber. It will be used as an equivalent to proximal edge, inflow edge, proximal portion, inflow portion.

Likewise “posterior edge” will be used to represent the distal edge of the ophthalmologic implant, i.e. the part of the body of the implant the farthest to the anterior chamber. It will be used as an equivalent to distal edge, outflow edge, distal portion, outflow portion.

The word “sub-scleral space” shall be understood as the anatomical space formed by the “supraciliary space” and the “suprachoroidal space”.

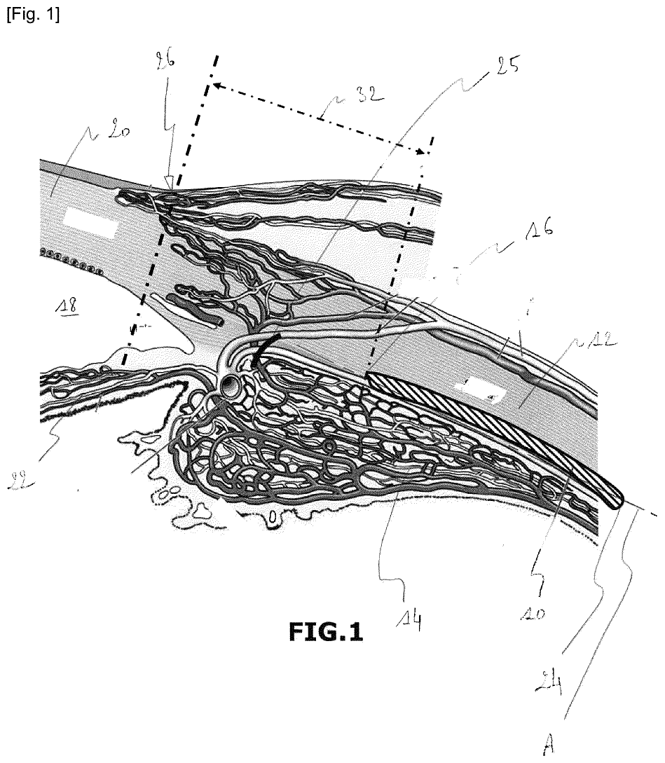

The present invention provides a method for inserting an ophthalmological implant 10 between the sclera 12 and an uveal tissue, here the ciliary body 14, in an eye of a patient. FIG. 1 shows an anatomical cross-section of an eye of a patient with an ophthalmological implant 10 in a final position after said method is performed.

The sclera 12 is connected to the periphery of the cornea 20 at the limbus 26. The sclera 12 overlies the ciliary body 14 which is connected to the iris 22. The iris root 25 is a part of the iris 22 which is fixed to the sclera 12, at the junction between the cornea 20 and the sclera 12. The anterior chamber 18 is disposed between the cornea 20 and the lens (not shown) bounded at its peripheral portion by the iris 22.

The purpose of the present method is to interpose the ophthalmological implant 10 between the sclera 12 and the ciliary body 14 at a specific position with regard to the limbus 26, called the final position.

In this method, an ophthalmological implant 10 is first provided. This implant 10 may be a cyclodialysis implant or a permanent interpositional ophthalmic implant. This ophthalmological implant 10 comprises an anterior edge 16 intended to face the anterior chamber 18 of the eye of the patient when disposed at its final position. The ophthalmological implant 10 also comprises a posterior edge 24 opposite the anterior edge 16 along an anteroposterior axis A.

The anteroposterior axis A extends radially with regard to the circle defined by the cornea 20, when observed in front of the cornea 20 or the eye of the patient.

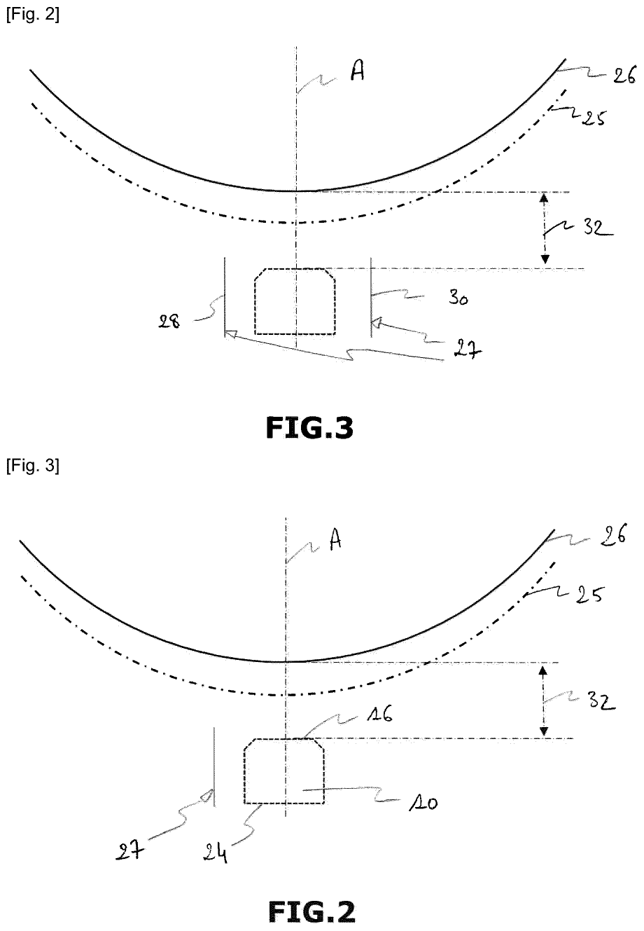

In this method, at least one incision 27 is made through the sclera 12 to form one or several scleral edges (see FIGS. 2 and 3). Said one or several scleral edges are then lifted and the ophthalmological implant 10 is inserted between the sclera 12 and the ciliary body 14 through said at least one incision 27.

The ophthalmological implant 10 is then positioned at a final position. Said final corresponds to an operational position of the ophthalmological implant 10 predetermined by the surgeon to reduce IOP. Particularly, this final position corresponds to the positional of the ophthalmological implant 10 when the surgical method is finished with the scleral edges naturally closed or sewn up.

This final position is set so that a distance 32 between the anterior edge 16 of the ophthalmological implant 10 and the limbus 26 of the eye of the patient is equal to or greater than 2 mm along the anteroposterior axis A. In other words, the anterior edge 16 of the ophthalmological implant 10 is distant from the limbus 26 to at least 2 mm along the anteroposterior axis A.

Alternatively or in combination, said distance 32 between the anterior edge 16 of the ophthalmological implant 10 and the limbus 26 of the eye of the patient is equal to or greater than 2.5 mm along the anteroposterior axis A. Said distance 32 may also be equal or greater than 3 mm.

This final position may be also set so that said distance 32 between the anterior edge 16 of the ophthalmological implant 10 and the limbus 26 of the eye of the patient is equal to or lower than 5 mm along the anteroposterior axis A.

Alternatively or in combination, said distance between the anterior edge of the ophthalmological implant and a limbus of the eye of the patient is equal to or lower than 4 mm along the anteroposterior axis. Said distance 32 may also be equal or lower than 3.5 mm.

FIGS. 2 and 3 show a partial frontal view of the eye of the patient. FIG. 2 shows a first embodiment of the method wherein only one incision 27 is made to insert the ophthalmological implant 10. FIG. 3 shows a second embodiment of the method wherein two incisions 27 are made to insert the ophthalmological implant 10. These two incisions 27 comprise a first incision 28 and a second incision 30. Providing at least two incisions 27 allows to insert the ophthalmological implant 10 through the first incision 28 while grabbing and pulling the ophthalmological implant 10 with a surgical tool through the second incision 30.

The incisions 27 extend along the anteroposterior axis A so that the implant is inserted through one incision 27 perpendicularly to the anteroposterior axis A.

Alternatively, the incisions 27 may extend perpendicularly to the anteroposterior axis A so that the ophthalmological implant 10 is inserted through one incision 27 along the anteroposterior axis A.

Furthermore, as shown on FIG. 2, said incision 27 is provided so that it faces said ophthalmological implant 10 when in the final position.

Alternatively, the incision 27 may be offset with regard to the final position of the ophthalmological implant 10. In this case, the incision 27 is provided at an intermediate position of the ophthalmological implant 10 so that said ophthalmological implant 10 is inserted through said incision 27 and then moved from said intermediate position wherein the ophthalmological implant 10 faces the incision 27 to said final position.

As shown on FIG. 3, said first 28 and second 30 incisions are provided so that said ophthalmological implant 10 is positioned between the first 28 and second 30 incisions when in the final position.

Alternatively, said first 28 and second 30 incisions may be offset with regard to the final position of the ophthalmological implant 10. Hence, these first 28 and second 30 incisions may be provided at an intermediate position of the ophthalmological implant 10 so that said ophthalmological implant 10 is inserted through said first 28 or second 30 incisions and then moved from said intermediate position wherein the ophthalmological implant 10 is between the first and second incisions to said final position.

In FIG. 3, said first 28 and second 30 incisions have the same longitudinal dimension. Alternatively, said first 28 and second 30 incisions may have different longitudinal dimensions.

This method may be used in combination to and/or after a conventional anti-glaucoma surgery or any intraocular procedure when a lowering of intraocular pressure is desirable. Conventional anti-glaucoma surgery may comprise trabeculectomy and sclerectomy which both require cutting of one or more scleral flaps from the eye that will be lifted to continue the procedure. A scleral flap is obtained by incising the sclera in one or two planes and to a variable depth, on three sides: two incisions substantially parallel to each other that extend from and away from the cornea 20 and a third incision perpendicular to the other two incisions and away from the cornea 20. After lifting the scleral flap(s), two incisions are made up to the ciliary body, inside and/or out of the flap, in order to slide the implant between the deep scleral plane and the ciliary body. The at least one incision 27 of the previously described method may be one or more of the incisions made in the conventional anti-glaucoma surgery. The step of making at least one incision and the step of inserting the implant can be performed in a single step with an injecting instrument.

1. A method for inserting an ophthalmological implant between a sclera and a ciliary body in an eye of a patient, comprising:
making at least one incision through the sclera in said eye of the patient forming at least one scleral edge,
inserting the ophthalmological implant between the sclera and the ciliary body through said at least one incision;
positioning the ophthalmological implant at a final position so that a distance between an anterior edge of the ophthalmological implant and a limbus of the eye of the patient is equal to or greater than 2 mm along an anteroposterior axis.

2. The method according to claim 1, wherein said distance between the anterior edge of the ophthalmological implant and the limbus of the eye of the patient is equal to or greater than 2.5 mm along the anteroposterior axis.

3. The method according to claim 1, wherein said distance between the anterior edge of the ophthalmological implant and a limbus of the eye of the patient is equal to or lower than 5 mm along the anteroposterior axis.

4. The method according to claim 3, wherein said distance between the anterior edge of the ophthalmological implant and a limbus of the eye of the patient is equal to or lower than 4 mm along the anteroposterior axis.

5. The method according to claim 1, wherein said ophthalmological implant has an anteroposterior dimension between the anterior and the posterior edges equal to or greater than 500 μm.

6. The method according to claim 1, wherein the inserting step comprises the injection of a viscoelastic fluid between the sclera and the uveal tissues.

7. The method according to claim 1, wherein said at least one incision extends along the anteroposterior axis.

8. The method according to claim 1, wherein said at least one incision comprises at least two incisions defining a first and a second incisions.

9. The method according to claim 8, wherein said first and second incisions are provided so that said ophthalmological implant is positioned between the first and second incisions when in the final position.

10. The method according to claim 8, wherein said first and second incisions are provided at an intermediate position of the ophthalmological implant so that said ophthalmological implant is inserted through said first or second incisions and then moved from said intermediate position wherein the ophthalmological implant is between the first and second incisions to said final position.

11. The method according to claim 1, wherein said method includes providing an ophthalmological implant having an anterior edge intended to face the anterior chamber and a posterior edge opposite the anterior edge.

12. The method according to claim 1, wherein said method includes lifting the at least one scleral edge prior to inserting the ophthalmological implant.
